(19) United States
(12) Patent Application Publication
(10) Pub. No.: US 2008/0228041 A1
(43) Pub. Date: Sep. 18, 2008

(54) Method and System for Remote Diagnosis and Supervision of Complex Procedures

(76) Inventor: David John Horrigan, Encinitas, CA (US)

Correspondence Address: HORRIGAN CORPORATION, 2300 EIGHTH ST., OLIVENHAIN, CA, 92024, US

(21) Appl. No.: 12/038,483

(22) Filed: Feb. 27, 2008

Related U.S. Application Data
(60) Provisional application No. 60/895,307, filed on Mar. 16, 2007.

(51) Int. Cl. G06F 19/00; A61B 5/00

(52) U.S. Cl. 600/300

(57) ABSTRACT

A network enabled method and system for better utilizing expert or licensed individuals to address or treat a greater quantity of situations at a lower cost and at greater convenience allowing numerous companies to have access to such expert or liscensed resources. This business model involves a trained assistant gathering appropriate data and transmitting it to a central or remote location where the specialist can review it and transmit a diagnosis or directions to the assistant to execute. The expert then collects the fees for the procedure and distributes proceeds to the assistant, assistants company or coordinating third party.

### CROSS-REFERENCE TO RELATED APPLICATIONS

Not Applicable

### SEQUENCE LISTING OF PROGRAM

Not Applicable

### FIELD OF THE INVENTION

The present invention relates to an expertise utilization method for business involved with remote diagnosis, direction and supervision from a liscensed or expert professional through the use of networked realtime communications such as instant messaging type communications that include remote instant voice, data, and video acquisition. The system allows the expert to determine which procedure to perform so that the remote assistant can perform the procedure and/or refer more complex situations to liscensed professionals. The method also allows that the diagnosing professional collects fees from insurance organizations and distributes proceeds to the assistant, assistant's company or coordinating third party. The system allows numerous companies to utilize a single remote expert and it allows the remote expert to address hundreds of cases per day as opposed to the few that he or she could normally attend to if clients had to come in to his or her office. This system makes use of high-speed data transfer via the internet, high quality; low bandwidth video and video-microscopy; and near-realtime communication utilizing SMS communications.

### BACKGROUND OF THE INVENTION

1. Prior Art

U.S. Pat. No. 6,882,825 System and method for providing help/training content for a web-based application

U.S. Pat. No. 6,868,395 Business transactions using the internet

U.S. Pat. No. 6,687,485 System and method for providing help/training content for a web-based application

U.S. Pat. No. 6,449,344 AOL IM

U.S. Pat. No. 7,123,695 Voice Delivery over IM

U.S. Pat. No. 7,152,093 multiple users

U.S. Pat. No. 7,056,217 Nintendo game IM

U.S. Pat. No. 7,152,093 Realtime IM with video

U.S. Pat. No. 7,162,436 Hotel selection

2. Description of Related Art

In present day medical offices and clinics it is common for medical assistants to be trained in complex procedures so that they can relieve the workload of the physician. These assistants then perform certain procedures under the close supervision of the doctor who being in the same office or clinic can be available should the assistant have questions or need closer supervision. In larger clinics it is not uncommon to have a dozen or more assistants for every physician on site.

In modern aesthetician and cosmetologist salons and spas the staff treat common cosmetic maintenance conditions but more and more these treatments border on complex medical situations. But the technicians are not qualified to distinguish between acne and dermatitis, for example, or aging nail symptoms and onychomycosis, a more complex condition.

Historically, medical professionals have diagnosed and advised remotely in emergency or highly specialized circumstances. The most obvious case is where emergency medical technicians first on the scene of an accident will hook an accident victim up to an EKG machine and send vital statistics to a nearby hospital for advice on how to treat before transporting. This predominantly radio-based system is limited by distance, the amount of data that it can send and whether or not there is a doctor available to receive and diagnose the data when sent. Though time is essentially the point of such technology, rarely are there enough accidents to justify a full-time specialist standing by just in case. Additionally, there are documented cases where individuals getting sick at sea were treated by courageous crew members while being advised by a doctor via short wave radio, but the limitations of this technique is that the doctor is blind to what's going on in his operating theatre and his remote assistants are almost always untrained assistants.

There are other documented cases where a specialist would participate in a complex medical procedure remotely via a closed circuit video feed. The problem with this system is that it is expensive to set up and not conducive to treating multiple cases simultaneously.

There are additionally a number of monitoring machines available that collect an individual's medical data and transmit it to a doctor via telephone or internet for monitoring special conditions and for avoiding emergencies. These methods are not particularly interactive nor are they oriented toward an improved business model for the physician. They essentially give the doctor a snapshot of vital statistics over a long period of time to be used when the patient comes in for a regular office visit.

There is an abundance of on-line education, sales and support methodologies, most of which are minimally interactive and not in realtime. The responses are relative to preprogrammed database data or email review, subject to the schedule of the professor or salesman or tech support expert.

### SUMMARY OF THE INVENTION

Accordingly it is a principal object of the present invention to overcome the disadvantages associated with prior art methods and systems and provide a simple business model to accomplish this. The invention is to better utilize specialized, expert or liscensed talent by having them supervise or advise numerous remote subscriber assistants from a central location using an instant communication network such as instant messaging technology along with its immediate two-way video, voice and data transfer via the internet or private network.

The present invention is advantageous in that it better utilizes specialized, expert or liscensed talent by having them supervise or advise numerous remote subscriber assistants from a central location.

It is also advantageous in that it allows the expert to bill for the procedure as an expert utilizing expert billing vehicles such as insurance while allowing a physician as an expert, for example, to see many more patients per day, albeit remotely.

The invention is also advantageous in that it allows small businesses utilizing trade assistants such as medical assistants to have expensive experts available instantly for a minimal cost. Not having to wait for the expert's schedule to accommodate the small businessperson's clients needs makes the invention a very desirable solution.

Another advantage of the invention is that it allows better utilization of specialized or licensed talent, letting them or their directees participate in numerous consults simultaneously. The remote agent saves them time as well, by attending to the minutia of the interview such as filling out pre-inspection interview questionnaires, testing, setting up cameras, microscopes and pre-interviewing or pre-qualifying subjects, patients or clients.

The present invention is further advantageous in that it allows the specialist to be exposed to many times the normal number of cases which will increase his exposure to anomalous cases which are beyond the abilities of the assistant but have special value to the specialist for treatment or referral.

Additionally, this invention allows the franchising of services to be delivered by lesser qualified individuals living in areas without such expert or professional talent immediately available.

### BRIEF DESCRIPTION OF THE DRAWINGS

For a better understanding of the business method invention with regard to the embodiments thereof, reference is made to the accompanying drawings, in which like numerals designate corresponding elements or sections throughout, and in which:

FIG. 1. illustrates a flow chart showing the elemental components of the present invention including an expert or liscensed professional, a remote assistant and a method for providing instant communication back and forth between the two parties.

FIG. 2. is a flow chart showing a group of expert or liscensed professionals, a remote assistant and a method for providing instant communication back and forth between the parties as well as the addition of an organization to manage the expert or liscensed professionals.

FIG. 3. is a flow chart showing the same network as in FIG. 2 but includes an organization to manage remote assistants or technicians.

FIG. 4. is a flow chart showing the same network as in FIG. 3 but with the addition of customers and with the addition of money flows illustrating who pays whom.

FIG. 5. is a flow chart as in FIG. 4 but with the omission of money flows and the addition of an anomalous client referral to a local expert or liscensed professional whose territory the assistant was servicing.

FIG. 6. is a flow chart as in FIG. 5 with the omission of the anomalous client referral to a local expert or liscensed professional and the addition of a referral to a local organization of assistants or technicians for the purpose of continuing to service a customer that the original assistant could no longer treat.

### DETAILED DESCRIPTION OF THE PREFERRED EMBODIMENT

As required, detailed embodiments of the present invention are disclosed herein, however, it is to be understood that the disclosed embodiments are merely exemplary of medical or cosmetic applications of the invention, which may be additionally embodied in various forms encompassing an expert diagnosis and supervision in different business requirements. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference, is now made to the drawings, wherein like characteristics and features of the pre-invention shown in the various FIGURES are designated by the same reference numerals.

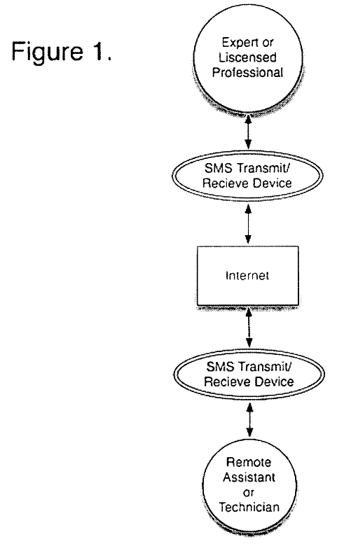

Referring to figures (1), (2), (3), (4), (5) and (6), illustrating the preferred embodiment for the invention: FIG. 1 is a flow chart showing a network made up of an assistant such as a medical assistant or medical technician, an expert or liscensed professional such as a doctor and a realtime long distance communication system such as an instant messaging system provided by AOL, CompuServe, or Apple Computer allowing both parties able to send and receive written, voice and video communication in real time. The invention allows that the assistant facilitates the doctor in gathering the necessary data for a diagnosis and then the doctcor directs the assistant as to the appropriate treatment procedure. It should be noted that in the case of medical or cosmetic treatment that video microscopy could be utilized remotely to diagnose the difference between fungal infections, bacterial infections and tumors.

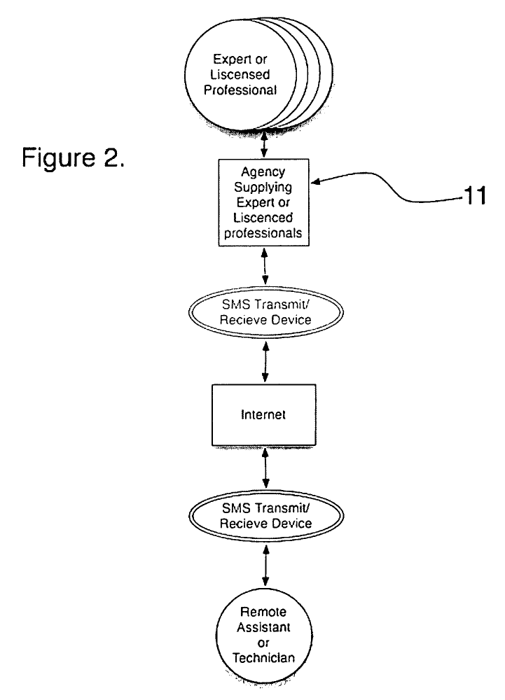

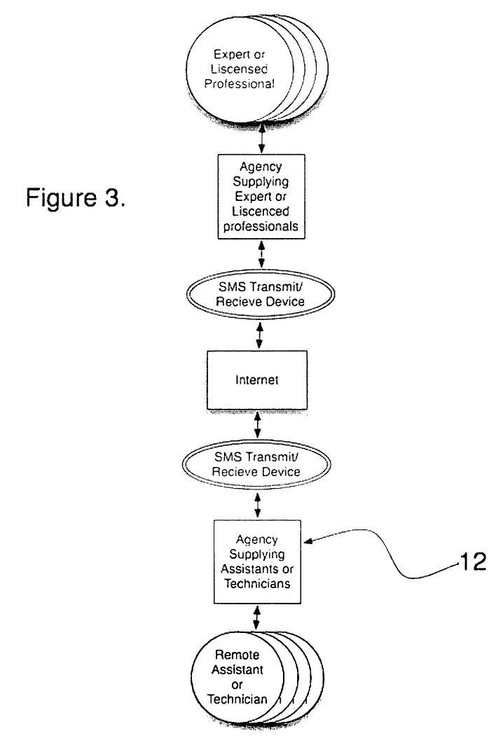

In FIG. 2 we have added an agency that would collect and organize professionals (FIG. 11) such as a clinic and better utilize them by having them do remote diagnosis for teams in the field or for remote clinics to the end of treating more patients in less time and providing diagnostic service where there was none before. FIG. 3 adds an agency for managing assistants or technicians (FIG. 12). This can be a spa chain with hundreds of cosmeticians or a single aesthetician being directed to clients from a manager. The end result is that they get professional diagnoses and direction to treat many more subjects more safely and more profitably.

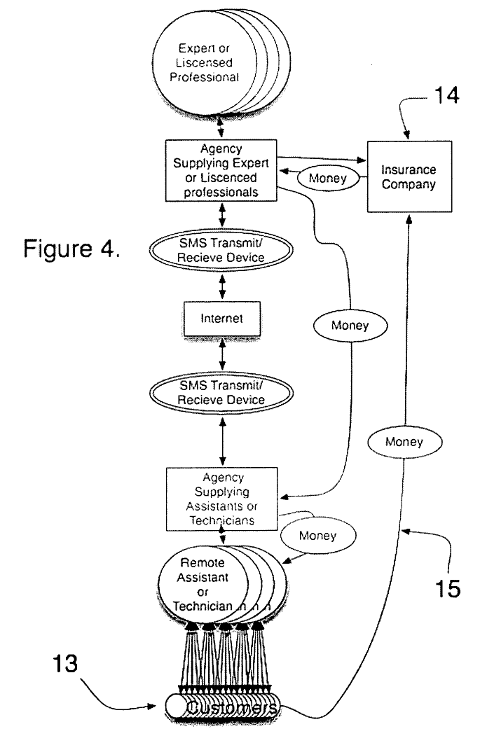

FIG. 4 illustrates the ultimate embodiment of the invention with a group of professionals such as doctors organized to be available for clinic or spa chains to instantly diagnose and if appropriate direct the trained staff of those clinics or spas so that they can more effectively treat an abundance of customers (FIG. 13). And these customers insurance companies (FIG. 14) can be billed directly by the expert or liscensed professional agency and the proceeds distributed appropriately (FIG. 15).

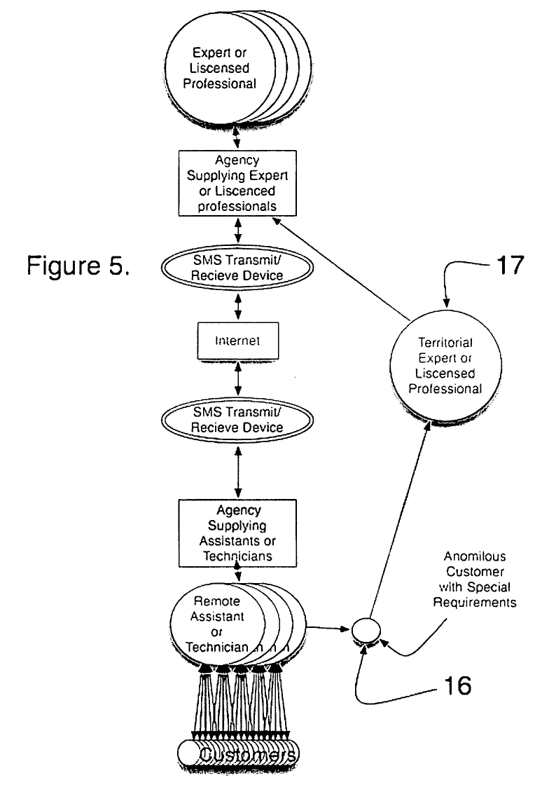

FIG. 5 illustrates the event that when a customer has special needs or requirements that are outside the capability of the assistant or technician, they can be referred to a local expert or liscensed professional for a fee or the local expert can franchise a territory.

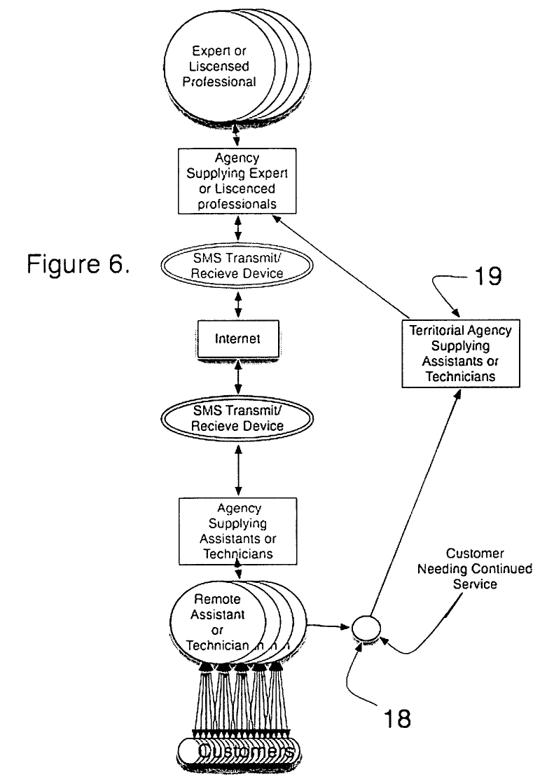

FIG. 6 illustrates the event that when an assistant organization has a client for a limited amount of time such as a vacation spa experience that the client can be referred to another assistant organization close to their home for continued treatment or service.

### Factors for Effectiveness

In order to make the invention as effective as possible there are two important factors that need to be taken into account. Firstly, the assistant needs to be thoroughly trained in all the appropriate diagnostic perception or information acquisition techniques such as by way of touch, smell, dissymmetry, and discoloration for example. Only so much information can be transmitted through video and video microscopy techniques so the deficiencies must be made up through exceptional attentiveness and through training.

Secondly, the liscensed professional on the diagnosing end of the system needs to be consistently and particularly diligent to detail and subtle indicators. The inclination when handling numerous remote patients or clients that one is not in direct communication with is to be inclined to handle them speedily and that speed is efficiency. In this model accuracy is efficiency and speed can result in missed diagnosis rendering the business method ineffective for its more advanced functions.

### SCOPE OF THE INVENTION

Thus the reader will see that this method and system creates an efficient network that supports medical technicians in the field or in remote offices allowing them the instant expertise derived from delivering visual data as well as other pertinent information to the distant doctor so that the doctors can, in return, give direction or oversee the technician's work or even issue prescriptions. While the above medical description contains many specificities, these should not be construed as limitations on the scope of my business model, but rather as an exemplification of one preferred embodiment thereof. Many other variations are possible. For example, the method could be applied to appraisers, attorneys, engineers, educators, or any number of professions where there is a shortage of liscensed professionals or experts and an abundance of patients, clients, or customers.

Accordingly, the scope of my business method should be determined, not by the embodiments illustrated, but by the appended claims and their legal equivalents.

I claim:

1. A system for remote diagnosis comprising:
a. an expert or liscensed professional remote from a practicing assistant,
b. a two-way realtime communication network connecting the expert and the assistant such as an instant messaging system equipped with suitable data acquisition and transfer devices working through said network to allow the expert to evaluate the remote situation and direct the assistant,
c. a practicing assistant qualified to exercise the directions of the expert and in need of the resources of the expert or licensed professional,
whereby the assistant services the client with more complex needs to the mutual benefit of all three parties.

2. A system for remote diagnosis as in claim 1 but with the addition of a team of experts organized under an agency whereby the agency can have experts available more readily.

3. A system for remote diagnosis as in claim 2 but with the addition of a team of assistants organized under an agency whereby the agency can have assistants available more readily.

4. A system for remote diagnosis as in claim 3 but with the addition that the expert agency does the billing for insurance reimbursement and forwards the due portion to the assistant agency.

5. A system for remote diagnosis as in claim 4 but with the addition that anomalous clients with special needs identified by the supervising expert can be referred to local experts or liscensed professionals.

6. A system for remote diagnosis as in claim 4 but with the addition that clients that need continued service beyond the availability of the assistant or their organization can be referred to assistant organizations in a preferred location.
